(19) United States
(12) Patent Application Publication
(10) Pub. No.: US 2019/0070159 A1
(43) Pub. Date: Mar. 7, 2019

(54) NOVEL PHARMACEUTICAL USES

(71) Applicant: TAKEDA PHARMACEUTICAL COMPANY LIMITED, Osaka (JP)

(72) Inventors: Richard JENKINS, London (GB); Mark HIBBERD, London (GB)

(21) Appl. No.: 16/178,335

(22) Filed: Nov. 1, 2018

Related U.S. Application Data
(63) Continuation of application No. 15/563,417, filed on Sep. 29, 2017, now abandoned.

(30) Foreign Application Priority Data
Mar. 31, 2015 (GB) 1505526.2
Nov. 27, 2015 (GB) 1521015.6

(51) Int. Cl. A61K 31/4439; A61K 45/06; A61K 31/4025; A61K 31/4184; A61K 31/506; A61K 31/5025; A61K 31/18

(52) U.S. Cl. CPC A61K 31/4439; A61K 2300/00; A61K 45/06; A61K 31/4025; A61K 31/18; A61K 31/506; A61K 31/5025; A61K 31/4184

(57) ABSTRACT

The invention provides a potassium-competitive acid blocker (P-CAB) for use in the treatment, prevention and/or reduction of gastro-esophageal reflux disease (GERD) symptoms in patients who are partial responders to a proton pump inhibitor (PPI). The P-CAB may, for example, be selected from 1-[5-(2-fluorophenyl)-1-(pyridin-3-ylsulfonyl)-1H-pyrrol-3-yl]-N-methylmethanamine (vonoprazan), revaprazan (YH1855), YH4808, RQ-4 and CS-526, or a salt thereof.

### CROSS-REFERENCE TO RELATED APPLICATIONS

This patent application is a continuation of U.S. patent application Ser. No. 15/563,417, filed Sep. 29, 2017, which is a National Stage of International Patent Application No. PCT/JP2016/061185, filed Mar. 30, 2016, which claims benefit of GB Application No. 1521015.6, filed Nov. 27, 2015 and GB Application No. 1505526.2, filed Mar. 31, 2015, the disclosure of each is hereby incorporated by reference in its entirety.

### FIELD OF THE INVENTION

The present invention relates to a novel use of a potassium-competitive acid blocker (P-CAB), and in particular, use thereof for the treatment, prevention and/or reduction of symptoms of gastro-esophageal reflux disease in patients having an insufficient (or partial) response to a proton pump inhibitor (PPI), for example in the treatment, prevention and/or reduction of symptoms of non-erosive reflux disease (NERD) and/or erosive esophagitis (EE).

### BACKGROUND OF THE INVENTION

Gastro-esophageal reflux disease (GERD) has been identified as the most common gastrointestinal diagnosis in outpatient clinics. Estimations suggest that up to 20% of adults are affected and over the last two decades there has been an overall increase in GERD, with trends indicating that further increases are expected.

The typical symptoms of GERD are heartburn and acid regurgitation and accompanying symptoms can also include epigastric pain, sleep disturbances, dyspepsia, dysphagia, odynophagia, nausea and vomiting. The main complications of GERD can be reflux esophagitis, the development of strictures, Barrett's esophagus (intestinal metaplasia and dysplasia) and esophageal adenocarcinoma. In rare cases, esophagitis may also lead to clinically significant bleeding and/or perforation.

The inhibition of gastric acid secretion is the cornerstone of the treatment of GERD, peptic ulcer, and other acid-related diseases. The development of histamine H₂ receptor antagonists (H₂RAs) in the 1970s represented the first major advancement in the treatment of acid-related diseases. However, H₂RAs have a relatively short duration of action, their effect on meal-stimulated acid secretion is weak, and their anti-secretory effect is diminished after repeated administration.

The development of proton pump inhibitors (PPIs) provided effective management for patients with erosive esophagitis (EE) and studies have shown that PPIs are superior to H₂RAs for the treatment of EE. PPIs have been extensively used clinically in a wide range of acid related disorders, including GERD, non-erosive esophagitis (NERD), peptic ulcer, nonsteroidal antiinflammatory drug (NSAID)-induced gastrointestinal injury and upper abdominal bleeding.

PPIs, such as omeprazole and lansoprazole, form a covalent bond with a cysteine residue of the enzyme H⁺/K⁺ ATPase and irreversibly inhibit the enzyme activity to suppress secretion of gastric acid. Although the efficacy of PPIs has been established, their efficacy can be improved in terms of acid stability and delayed onset of action and several researchers have attempted to develop new classes of novel pharmaceutical agents for the treatment of GERD. These agents include transient lower esophageal sphincter relaxation-reducing agents, serotonergic agents/prokinetics, mucosal protectants, histamine H₃ agonists and anti-gastrin agents.

In addition, an important new class of acid suppressant, the potassium-competitive acid blocker (P-CAB), has been developed that inhibit proton pump (H⁺/K⁺ ATPase) activity reversibly and in a K⁺ antagonist inhibitory manner. P-CAB compounds that have been studied include soraprazan (BY359), revaprazan (YH1885), AZD0865, YH4808, SCH 28080, CS-526 and vonoprazan (TAK-438). These agents bind ionically to the proton pump at or near the potassium-binding site in a K⁺ competitive manner, thereby blocking acid secretion through a direct, reversible mechanism. P-CABs have higher pKₐ values than PPIs, and they are stable at low pH. These properties allow P-CABs to become highly concentrated in the strongly acidic compartment of the gastric parietal cell at the luminal surface of the H⁺/K⁺ ATPase and to exert a less variable onset of their effect. They have a rapid onset of action (within 1 day of dosing) due to P-CAB superconcentration within the parietal cell canaliculus, and a luminal site of action. It has been reported that the P-CABs produced equivalent or superior inhibition of acid secretion compared with a PPI in various animal studies. (Vakil, Aliment. Pharmacol. Ther., 19, 1041, 2004).

Vonoprazan (TAK-438) is a novel, orally active small-molecule P-CAB which has been shown in both single and multiple repeat-dosing studies to have a rapid onset of action after the first dose and near maximal effect on pH holding time within 24 hours of dosing, which is maintained with chronic dosing (Nishida et al., Bioorg. & Med. Chem. 20, 3925, 2012). In one study, the results indicated that TAK-438 exerts a more potent and longer-lasting inhibitory action on gastric acid secretion in rats than the PPI, lansoprazole (Hori et al. J. Pharm. Exp. Therapeutics, 335, 231, 2010). The reported data are not, however, predictive of clinical effects in humans, and furthermore actually suggest that the in vivo differences between TAK-438 and a PPI are difficult to predict, even in rats, based on in vitro antisecretory effect data. In addition, clinical data in patients with erosive esophagitis (EE) demonstrated that TAK-438 was effective for the treatment of EE and maintenance of EE healing (Iwakiri et al., Gastroenterol., vol. 146(5)(Suppl. 1): S-741, 2014; Umegaki et al., Gastroenterol, vol. 146(5)(Suppl. 1): S-738, 2014). However, the data presented did not provide any distinction between patients with different levels of PPI-responsiveness.

Another P-CAB, AZD0865, has been shown to be a gastric acid-suppressing agent that has a rapid onset of action and long duration of effect. The efficacy and safety of AZD0865 in the treatment of patients with non-erosive reflux disease (NERD) has been investigated in a study comprising patients with troublesome heartburn for at least 6 months and no evidence of erosions. The patients were randomized to receive AZD0865 or esomeprazole for 4 weeks. Throughout the treatment period, patients reported the presence and intensity of heartburn and other NERD symptoms twice daily using an electronic diary. The results of the study showed that although the P-CAB, AZD0865, displayed a rapid inhibition of acid production and had a prolonged, dose-dependent duration of effect it did not provide clinical benefit over esomeprazole in the treatment of the symptoms of patients with NERD. (Dent et al., American Journal of Gastroenterology 103, 20-26, 2008).

It has also been suggested that increasing the degree of acid inhibition beyond that already achieved by either a PPI or a higher dose of the P-CAB does not translate into increased clinical efficacy in esophagitis patients. (Kahrilas et al., Clinical Gastroenterology & Hepatology, 5, 1385-1391, 2007).

A recent review on the management of patients with an incomplete response to PPI therapy discussed and suggested a number of alternative approaches for the treatment of GERD symptoms for these patients (Kahrilas et al, Best Practice & Research Clin. Gastroenterology, vol. 27, 401-414, 2013). However, the review concluded that the development of new classes of compounds, such as P-CABs, acting through novel mechanisms to reduce acid secretion, was unlikely to improve symptom control beyond that achieved with PPIs because a P-CAB, capable of nearly complete and immediate acid inhibition, had not been shown to be superior to a PPI with respect to symptom control in patients with either erosive or non-erosive reflux disease.

Studies have so far failed to demonstrate that the theoretical advantages related to superior pharmacodynamic properties of acid inhibition by P-CABs translates into improved efficacy in the treatment of GERD patients as studies have shown that P-CABs are similar in the magnitude of treatment effect and did not provide greater efficacy than PPIs in terms of the degree of GERD symptom control.

A review of the prior art suggests that there exists a plateau effect for acid secretion inhibition with respect to symptom control and increasing the inhibition of acid secretion by either increasing the PPI dose or by using more powerful acid secretion inhibitors, such as P-CABs, would not be expected to be more effective. Consequently, the prior art teaches that alternative strategies, i.e. other than further increasing acid secretion inhibition, should be explored for the control of GERD symptoms. For example, it has been suggested that increasing the duration of the gastric acid blocking response, e.g. using controlled release PPI formulations, may be an appropriate strategy (Scarpignato, Neurogastroenterol. Motil., vol. 24, 697-704, 2012).

In recent years, there have been a growing number of reports suggesting that about 30% of GERD patients treated with PPI are partially or completely unresponsive to standard dose and duration of PPI therapy. For these patients, it is usually suggested—as a first step—to increase (usually double) the dose and duration of therapy with a PPI. Alternatively, the patient can be switched to another PPI. In this connection, it has also been found that patients exposed to higher doses of PPIs, particularly over longer periods, may suffer an increased risk of osteoporosis-related fractures (T. Ito and R. T Jensen, Curr Gastroenterol Rep., vol. 12(6), 448-457, 2010; and Nexium© (esomeprazole magnesium) US FDA Prescribing Information, Ref ID 3675799).

Diagnostic evaluation of patients with GERD who have failed PPI treatment may include an upper endoscopy, pH testing and esophageal impedance with pH monitoring. Commonly, doubling the PPI dose or switching to another PPI will be pursued by the treating physician but the failure of such a therapeutic strategy may result in the addition of a transient lower esophageal sphincter reducer or pain modulator. Alternatively, anti-reflux surgery may be suitable for a subset of patients.

Failure of PPIs to resolve symptoms in GERD patients can also lead to a number of additional deleterious effects on the patients, some of which are non-gastrointestinal. These effects include nighttime awakenings and insomnia, fatigue associated with sleep disturbance due to GERD, daytime sleepiness associated with sleep disturbance, accidents associated with sleep disturbance, increased physician visits and hospital admissions, reduction in physical and mental functioning, including concentration, anxiety, increased use of other medications for the treatment of GERD, and reduction in general wellbeing and Quality of Life.

The European Medicines Agency (EMA) have recognized partial response to a PPI as a medical issue. The 2011 revision of the “Guideline on the evaluation of drugs for the treatment of Gastro-esophageal reflux disease” includes recommendations on how to assess PPI partial responders (patients with an insufficient response to a PPI).

PPI Partial responders are defined by the presence of both heartburn and acid regurgitation at the time of primary diagnosis. Partial response should also be based on medical history, indicating a reduction in typical symptoms with an adequate course of PPI therapy. On cessation of PPI therapy, PPI partial responders would be expected to experience a worsening in GERD symptoms. For example, a class of PPI partial responders will, at diagnosis, have a history of eight or more weeks of persistent heartburn and/or acid regurgitation (e.g. symptoms on two or more days a week), despite appropriate treatment with a standard course of PPI therapy. Also for example, a class of PPI partial responders will have a history of heartburn on two to five days and acid regurgitation on one or more days during the final week of a four-week PPI treatment period (using, for example, esomeprazole 40 mg, once-a-day), and an increase of two or more heartburn symptom days (i.e. a total of four to seven symptom days) and at least one acid regurgitation symptom day in the final week of a subsequent two-week period of placebo administration (i.e. no PPI administration).

The typical symptoms of GERD are considered to be heartburn and acid regurgitation. GERD is a symptom-driven disease that is normally evaluated based on the presence, frequency, and severity of GERD symptoms.

Despite considerable advances in drug treatment over the past two decades, patients receiving medication for the treatment of GERD continue to have a number of important needs for more effective control of their symptoms. In view of the importance of this unmet medical need, there is a great need for developing an effective treatment for the reduction of GERD symptoms in this difficult-to-treat population of patients with an insufficient response to a PPI.

### SUMMARY OF THE INVENTION

A first aspect of the present invention provides a potassium-competitive acid blocker (P-CAB) for use in the treatment, prevention and/or reduction of GERD symptoms in patients with an insufficient response to a proton pump inhibitor (PPI) (i.e. PPI partial responders).

A second aspect of the present invention provides a P-CAB for use in the treatment, prevention and/or reduction of symptoms of non-erosive reflux disease (NERD) or erosive esophagitis (EE) of Grade A as defined by the Los Angeles (LA) Classification.

A third aspect of the present invention provides a pharmaceutical composition comprising the P-CAB for use according to the first or second aspect of the invention.

In a fourth aspect, the present invention provides a method of treating, preventing and/or reducing GERD symptoms in a patient in need thereof, wherein the patient has an insufficient response to a proton pump inhibitor (PPI) (i.e. PPI partial responder), the method comprising administering to the patient a prophylactically or therapeutically effective amount of a potassium-competitive acid blocker (P-CAB).

The present invention also provides the use of a potassium-competitive acid blocker (P-CAB) for the manufacture of a medicament for use in the treatment, prevention and/or reduction of GERD symptoms in patients with an insufficient response to a proton pump inhibitor (PPI) (i.e. PPI partial responders).

The present invention also provides the use of a P-CAB for the manufacture of a medicament for use in the treatment, prevention and/or reduction of symptoms of non-erosive reflux disease (NERD) or erosive esophagitis (EE) of Grade A as defined by the Los Angeles (LA) Classification.

Preferably the P-CAB for use according to the above aspects of the present invention is for the treatment, prevention and/or reduction of symptoms of non-erosive reflux disease (NERD).

Preferably the P-CAB for use according to the above aspects of the present invention is for the treatment, prevention and/or reduction of symptoms of erosive esophagitis (EE) of Grade A as defined by the Los Angeles (LA) Classification.

Preferably the P-CAB for use according to the above aspects of the present invention is for the sustained reduction of GERD symptoms in patients with an insufficient response to a PPI.

Preferably the P-CAB for use according to the above aspects of the present invention is for the reduction in nighttime awakenings and insomnia associated with GERD.

Preferably the P-CAB for use according to the above aspects of the present invention is for the reduction in fatigue associated with sleep disturbance due to GERD.

Preferably the P-CAB for use according to the above aspects of the present invention is for the reduction in daytime sleepiness associated with sleep disturbance due to GERD.

Preferably the P-CAB for use according to the above aspects of the present invention is for the reduction in the patient's accidents associated with sleep disturbance due to GERD.

Preferably the P-CAB for use according to the above aspects of the present invention is for the reduction in the patient's physician visits and hospital admissions associated with GERD.

Preferably the P-CAB for use according to the above aspects of the present invention is for the improvement in physical and mental functioning, including concentration, in patients with GERD.

Preferably the P-CAB for use according to the above aspects of the present invention is for the reduction in anxiety associated with GERD.

Preferably the P-CAB for use according to the above aspects of the present invention is for the reduction in the patient's use of other medications for the treatment of GERD.

Preferably the P-CAB for use according to the above aspects of the present invention is for the improvement of general wellbeing and Quality of Life in patients with GERD.

Preferably the P-CAB for use according to the above aspects of the present invention is for the reduction of inflammation in patients with GERD.

For the avoidance of doubt, it should be noted that all particular and/or preferred embodiments identified herein are applicable to both the ‘use’ and ‘method’ aspects defined above. Moreover, it will be appreciated that the various additional applications/embodiments of the use and method of the invention mentioned above (from reduction in nighttime awakenings and insomnia through to improvement of general wellbeing and Quality of Life, and the reduction of inflammation) can also be considered advantages of the invention.

In an embodiment of the fourth aspect of the invention, the method of treating, preventing and/or reducing GERD symptoms in a patient in need thereof comprises the step of:
a) identifying a patient suffering from GERD symptoms, wherein said patient has been administered, is being administered or is about to be administered a PPI.

In such an embodiment, the method may further comprise the step of:
b) preferentially selecting one or more particular P-CABs (preferably vonoprazan) from a group of treatment options for symptoms of GERD (such as P-CABs) to administer to the patient.

Furthermore, such an embodiment may additionally comprise the step of:
c) administering to said patient a therapeutically effective amount of the selected P-CAB(s) (preferably vonoprazan) to treat, prevent and/or reduce the GERD symptoms.

Preferably, the P-CAB for use according to the present invention is selected from 1-[5-(2-fluorophenyl)-1-(pyridin-3-ylsulfonyl)-1H-pyrrol-3-yl]-N-methylmethanamine (vonoprazan, TAK-438), revaprazan (YH1855), YH4808 (Yuhan Corporation), RQ-4 (also known as RQ-00000004; RaQualia Pharma Inc.), RQ-774 (also known as RQ-00000774; RaQualia Pharma Inc.) and CS-526 (Daiichi Sankyo), and salts thereof (in particular, pharmaceutically acceptable salts). More preferably, the P-CAB is vonoprazan or a pharmaceutically acceptable salt thereof.

A particularly preferred P-CAB is vonoprazan fumarate.

P-CABs suitable for use according to the present invention are also disclosed, for example, in EP-A-1784404, including the following:
1-benzyl-7-[N-(4-fluorobenzyl)-N-methyl]amino-2,3-dimethyl-1H-pyrrolo[2,3-c]pyridine hydrochloride;
1-(3-fluorobenzyl)-7-(4-fluorobenzylamino)-2,3-dimethyl-1H-pyrrolo[2,3-c]pyridine hydrochloride;
7-(4-fluorobenzylamino)-1-isobutyl-2,3-dimethyl-1H-pyrrolo[2,3-c]pyridine hydrochloride;
11-isobutyl-2,3-dimethyl-7-(2-methylbenzylamino)-1H-pyrrolo[2,3-c]pyridine hydrochloride;
7-(4-chlorobenzylamino)-1-(4-fluorobenzyl)-2,3-dimethyl-1H-pyrrolo[2,3-c]pyridine hydrochloride;
7-(4-chlorobenzylamino)-2,3-dimethyl-1-propyl-1H-pyrrolo[2,3-c]pyridine hydrochloride;
2,3-dimethyl-7-(naphthalen-2-yl)-1H-pyrrolo[2,3-c]pyridine hydrochloride;
7-(1,2,3,4-tetrahydroisoquinolin-2-yl)-2,3-dimethyl-1H-pyrrolo[2,3-c]pyridine hydrochloride;
1-benzyl-7-(1,2,3,4-tetrahydroisoquinolin-2-yl)-2,3-dimethyl-1H-pyrrolo[2,3-c]pyridine hydrochloride;
7-(1,2,3,4-tetrahydroisoquinolin-2-yl)-1,2,3-trimethyl-1H-pyrrolo[2,3-c]pyridine hydrochloride;
1-propyl-7-(1,2,3,4-tetrahydroisoquinolin-2-yl)-2,3-dimethyl-1H-pyrrolo[2,3-c]pyridine hydrochloride;
1-(2-methylthiazol-4-ylmethyl)-7-(1,2,3,4-tetrahydroisoquinolin-2-yl)-2,3-dimethyl-1H-pyrrolo[2,3-c]pyridine hydrochloride;
1-(4-methylbenzyl)-7-(1,2,3,4-tetrahydroisoquinolin-2-yl)-2,3-dimethyl-1H-pyrrolo[2,3-c]pyridine hydrochloride;
1-(3-fluorobenzyl)-7-(1,2,3,4-tetrahydroisoquinolin-2-yl)-2,3-dimethyl-1H-pyrrolo[2,3-c]pyridine hydrochloride;
1-(4-methoxybenzyl)-7-(1,2,3,4-tetrahydroisoquinolin-2-yl)-2,3-dimethyl-1H-pyrrolo[2,3-c]pyridine hydrochloride;
1-allyl-7-(1,2,3,4-tetrahydroisoquinolin-2-yl)-2,3-dimethyl-1H-pyrrolo[2,3-c]pyridine hydrochloride;
1-(4-tert-butylbenzyl)-7-(1,2,3,4-tetrahydroisoquinolin-2-yl)-2,3-dimethyl-1H-pyrrolo[2,3-c]pyridine hydrochloride;
1-(2-vinyloxyethyl)-7-(1,2,3,4-tetrahydroisoquinolin-2-yl)-2,3-dimethyl-1H-pyrrolo[2,3-c]pyridine hydrochloride;
2-(1-allyl-2,3-dimethyl-1H-pyrrolo[2,3-c]pyridin-7-yl)-1-methyl-1,2,3,4-tetrahydroisoquinoline hydrochloride;
2-[1-(3-fluorobenzyl)-2,3-dimethyl-1H-pyrrolo[2,3-c]pyridin-7-yl]-1-methyl-1,2,3,4-tetrahydroisoquinoline hydrochloride; and
2-(1-allyl-2,3-dimethyl-1H-pyrrolo[2,3-c]pyridin-7-yl)-6-fluoro-1-methyl-1,2,3,4-tetrahydroisoquinoline hydrochloride, and suitable alternative salts thereof.

In particular, 1-benzyl-7-(1,2,3,4-tetrahydroisoquinolin-2-yl)-2,3-dimethyl-1H-pyrrolo[2,3-c]pyridine hydrochloride; and 1-(3-fluorobenzyl)-7-(1,2,3,4-tetrahydroisoquinolin-2-yl)-2,3-dimethyl-1H-pyrrolo[2,3-c]pyridine hydrochloride (and suitable alternative salts thereof) may be mentioned.

In addition, P-CABs suitable for use according to the present invention are disclosed, for example, in U.S. Pat. No. 8,648,080, including the following:
(S)-(−)-4-[(5,7-difluoro-3,4-dihydro-2H-chromen-4-yl)oxy]-N,N,2-trimethyl-1H-benzimidazole-6-carboxamide;
(S)-(−)-4-[(5,7-difluoro-3,4-dihydro-2H-chromen-4-yl)oxy]-2-methyl-6-(pyrrolidin-1-ylcarbonyl)-1H-benzimidazole;
(S)-(−)-4-[(5-fluoro-3,4-dihydro-2H-chromen-4-yl)oxy]-N,N,2-trimethyl-1H-benzimidazole-6-carboxamide;
(−)-1-(2-methoxyethyl)-N,N,2-trimethyl-8-phenyl-1,6,7,8-tetrahydrochromeno[7,8-d]imidazole-5-carboxamide;
(+8-(4-fluorophenyl)-1-(2-methoxyethyl)-N,N,2-trimethyl-1,6,7,8-tetrahydrochromeno[7,8-d]imidazole-5-carboxamide;
8-(4-fluorophenyl)-1-(3-hydroxypropyl)-N,N,2-trimethyl-1,6,7,8-tetrahydrochromeno[7,8-d]imidazole-5-carboxamide;
8-(4-fluorophenyl)-1-(isoxazol-3-ylmethyl)-N,N,2-trimethyl-1,6,7,8-tetrahydrochromeno[7,8-d]imidazole-5-carboxamide;
8-(4-fluorophenyl)-N-(2-hydroxyethyl)-1-(2-methoxyethyl)-N,2-dimethyl-1,6,7,8-tetrahydrochromeno[8,7-d]imidazole-5-carboxamide;
(8-(4-fluorophenyl)-1-(2-methoxyethyl)-2-methyl-1,6,7,8-tetrahydrochromeno[8,7-d]imidazol-5-yl)(morpholino)methanone;
1-{5-(2-fluorophenyl)-1-[(6-methylpyridin-3-yl)sulfonyl]-1H-pyrrol-3-yl}-N-methylmethanamine;
1-[4-fluoro-5-phenyl-1-(pyridin-3-ylsulfonyl)-1H-pyrrol-3-yl]-N-methylmethanamine;
N-methyl-1-[5-(4-methyl-3-thienyl)-1-(pyridin-3-ylsulfonyl)-1H-pyrrol-3-yl]methanamine;
1-[5-(2-fluorophenyl)-1-(pyridin-3-ylsulfonyl)-1H-pyrrol-3-yl]-N-methylmethanamine;
N-Methyl-1-[5-(2-methylphenyl)-1-(pyridine-3-ylsulfonyl)-1H-pyrrol-3-yl]methanamine or;
8-[{2,6-dimethylbenzyl}amino]-N-[2-hydroxyethyl]-2,3-dimethylimidazo[1,2-a]pyridine-6-carboxamide;
8R,9R)-7-(2-methoxyethoxy)-2,3-dimethyl-9-phenyl-7,8,9,10-tetrahydroimidazo[1,2-h][1,7]naphthyridine-8-ol;
5,6-dimethyl-2-(4-fluoro-phenylamino)-4-(1-methyl-1,2,3,4-tetrahydroisoquinoline-2-yl)pyrimidine;
(S)-(−)-N,N,2,3-tetramethyl-8-O-tolyl-3,6,7,8-tetrahydrochromeno[7,8-d]imidazole-5-carboxamide; and
7-(4-fluorobenzyloxy)-2,3-dimethyl-1-{[(1 S,2S)-2-methyl cyclopropyl]methyl}-1H-pyrrolo[2,3-d]pyridazine,
   and suitable salts thereof.

In particular, (S)-(−)-4-[(5,7-difluoro-3,4-dihydro-2H-chromen-4-yl)oxy]-N,N,2-trimethyl-1H-benzimidazole-6-carboxamide; (−)-1-(2-methoxyethyl)-N,N,2-trimethyl-8-phenyl-1,6,7,8-tetrahydrochromeno[7,8-d]imidazole-5-carboxamide; and 8-[{2,6-dimethylbenzyl}amino]-N-[2-hydroxyethyl]-2,3-dimethylimidazo[1,2-a]pyridine-6-carboxamide (and suitable salts thereof) may be mentioned.

Preferably, the P-CAB for use according to the present invention is administered at a dose of the P-CAB of 0.5 mg to 500 mg per day. Preferably the dose is 10 mg to 200 mg per day and more preferred is 20 mg to 40 mg per day. A particularly preferred dose of the P-CAB is 20 mg per day. Another particularly preferred dose of the P-CAB is 40 mg per day. Another preferred dose of the P-CAB (e.g. TAK-438) is 10 mg per day. In preferred embodiments, the P-CAB is administered once per day (i.e. once daily dosing).

Also preferable is that the P-CAB is used in a composition which is of the conventional-release type. In other words, the composition is of the instant release/non-extended/non-sustained/non-controlled release type.

### DETAILED DESCRIPTION OF THE INVENTION

The present invention has surprisingly revealed that potassium-competitive acid blockers (P-CABs) may be effective for the treatment, prevention and/or reduction of symptoms of non-erosive reflux disease (NERD) and/or erosive esophagitis (EE) of Grade A as defined by the Los Angeles (LA) Classification.

The present invention has also surprisingly revealed that potassium-competitive acid blockers (P-CABs) may be effective for the treatment, prevention and/or reduction of GERD symptoms in the difficult to treat population of patients with an insufficient (partial) response to a proton pump inhibitor (PPI). Such a marked difference in efficacy compared to the use of PPIs in this patient population is surprising, given the teaching of the prior art that the additional acid secretion inhibition capability of P-CABs does not necessarily translate into enhanced symptom control.

The definition of PPI non-responders (PPI refractory patients, PPI resistant patients, PPI failure patients), who have no response at all to PPIs, is different to the definition of patients who have an insufficient response to a PPI. Patients with an insufficient response to a PPI are also called partial responders, as they have a partial but not full response to the PPIs and their symptoms worsen if they stop taking the PPI.

Proton pump inhibitors (PPIs) are defined as compounds that can form a covalent bond with a cysteine residue of the enzyme H⁺/K⁺ ATPase and irreversibly inhibit the enzyme activity.

Examples of PPIs are lansoprazole, pantoprazole, omeprazole, rabeprazole or an optically active form thereof, such as dexlansoprazole or esomeprazole, or a salt thereof.

A potassium-competitive acid blocker (P-CAB) is defined as a compound that inhibits H⁺/K⁺ ATPase activity reversibly and in a K⁺ antagonist inhibitory manner. P-CABs bind ionically to H⁺/K⁺ ATPase enzyme at or near the potassium-binding site in a K⁺ competitive manner, thereby blocking acid secretion through a direct, reversible mechanism.

Examples of P-CAB compounds for use in the present invention include revaprazan (YH1885), YH4808, vonoprazan (TAK-438), RQ-4 and CS-526 and their pharmaceutically acceptable salts.

A particularly preferred P-CAB for use in the present invention is vonoprazan (TAK-438), or a pharmaceutically acceptable salt thereof. A particularly preferred compound is vonoprazan fumarate (see Formula I).

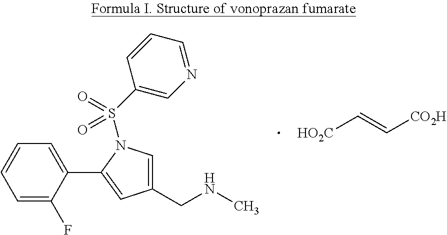 [SMILES: CNCc1cc(-c2ccccc2F)n(S(=O)(=O)c2cccnc2)c1 ; O=C(O)/C=C/C(=O)O]

Preferably, the vonoprazan or its pharmaceutically acceptable salt, in particular the fumarate salt, for use according to the present invention is administered at a dose of 0.5 mg to 120 mg per day, more preferably 10 mg to 50 mg per day and even more preferably 20 mg to 40 mg per day. A particularly preferred dose is 40 mg per day. Another particularly preferred dose is 20 mg per day. Where dose amounts of vonoprazan (TAK-438) are specified herein, this refers to the amount of vonoprazan free base in the dose. Thus, where a vonoprazan salt is to be used, the overall dose amount of the salt will be higher, as would be appreciated by the skilled person.

A particular embodiment employs non-extended release tablets of TAK-438 (vonoprazan) containing 40 mg (with respect to free base) per tablet TAK-438 as its fumarate salt.

The definition of EE used herein is based on the ‘LA classification’. The Los Angeles (LA) Classification of Esophagitis is the most widely used system to describe the endoscopic appearance of reflux esophagitis and grade its severity, and uses the following classifications: (Dent, Best Practice & Research Clinical Gastroenterology, Vol. 22, No. 4, pp. 585-599, 2008)

Grade A—One (or more) mucosal break no longer than 5 mm that does not extend between the tops of two mucosal folds.

Grade B—One (or more) mucosal break more than 5 mm long that does not extend between the tops of two mucosal folds.

Grade C—One (or more) mucosal break that is continuous between the tops of two or more mucosal folds but which involve less than 75% of the circumference.

Grade D—One (or more) mucosal break which involves at least 75% of the esophageal circumference.

The actual PPI to which the patient is a partial responder is not limited in any way, but the PPI is typically selected from lansoprazole, pantoprazole, omeprazole, rabeprazole or an optically active form thereof, such as dexlansoprazole or esomeprazole, or a salt thereof. Preferably the PPI is esomeprazole or a salt thereof.

In an embodiment of the use and method of the invention, the P-CAB administration results in an improvement in heartburn-free days which is at least 1% greater than that achieved by PPI administration in subjects who are PPI partial responders. In another embodiment, the improvement in heartburn free days is at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 40%, at least 50%, at least 60%, at least 70%, at least 80%, at least 90% or at least 100%.

In another embodiment of the use and method of the invention, the P-CAB administration results in an improvement in heartburn symptoms which is at least 1% greater than that achieved by PPI administration in subjects who are PPI partial responders In another embodiment, the improvement in heartburn symptoms is at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 40%, at least 50%, at least 60%, at least 70%, at least 80%, at least 90% or 100%.

In yet another embodiment of the use and method of the invention, the P-CAB administration results in an improvement in regurgitation symptoms which is at least 1% greater than that achieved by PPI administration in subjects who are PPI partial responders. In another embodiment, the improvement in regurgitation symptoms is at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 40%, at least 50%, at least 60%, at least 70%, at least 80%, at least 90% or 100%.

The pharmaceutical composition according to the third aspect of the present invention can be a solution or suspension, but is preferably a solid oral dosage form. Preferred solid oral dosage forms in accordance with the invention include tablets, capsules and the like which, optionally, may be coated if desired. Tablets can be prepared by conventional techniques, including direct compression, wet granulation and dry granulation. Capsules are generally formed from a gelatine material and can include a conventionally prepared granulate of excipients.

The pharmaceutical composition according to the third aspect of the present invention typically comprises one or more conventional pharmaceutically acceptable excipient(s) selected from the group comprising a filler, a binder, a disintegrant, a lubricant and optionally further comprises at least one excipient selected from colouring agents, adsorbents, surfactants, film formers and plasticizers. A number of suitable compositions containing vonoprazan (TAK-438), as an exemplary P-CAB, are disclosed in WO 2010/013823.

The P-CAB for use according to the present invention can be appropriately administered orally or parenterally (e.g., topical, rectal, intravenous administrations and the like) as it is or as a pharmaceutical composition containing a pharmacologically acceptable carrier admixed according to a method known per se, such as tablets (including sugar-coated tablets and film-coated tablets), powder, granule, capsule (including soft capsule), orally disintegrating tablet, orally disintegrating film, liquid, injection, suppository, sustained- or (preferably) immediate/instant-release preparation, plaster and the like. Particularly, the P-CAB for use of the present invention is preferably administered as an oral pharmaceutical composition in the form of tablet, granule, capsule and the like.

The content of P-CAB in the pharmaceutical composition of the present invention is about 0.01 to 100% by weight relative to the entire composition. Though subject to change depending on the administration target, administration route, target disease and the like, the dose of the P-CAB is preferably 0.5 to 500 mg per day, more preferably 5 to 500 mg per day, even more preferably 10 to 200 mg per day, such as 20 to 40 mg per day, based on the active ingredient and may be administered once daily or in 2 or more divided portions per day. In preferred embodiments, the P-CAB is administered once daily.

The pharmacologically acceptable carrier that may be used to produce the pharmaceutical composition of the present invention includes various organic or inorganic carrier substances in common use as pharmaceutical materials, including excipients, lubricants, binders, disintegrants, water-soluble polymers and basic inorganic salts for solid preparations; and solvents, dissolution aids, suspending agents, isotonizing agents, buffers and soothing agents for liquid preparations and the like. Other ordinary pharmaceutical additives such as preservatives, anti-oxidants, coloring agents, sweetening agents, souring agents, bubbling agents and flavorings may also be used as necessary.

Such “excipients” include, for example, lactose, sucrose, D-mannitol, starch, cornstarch, crystalline cellulose, light silicic anhydride, titanium oxide and the like. Such “lubricants” include, for example, magnesium stearate, sucrose fatty acid esters, polyethylene glycol, talc, stearic acid and the like. Such “binders” include, for example, hydroxypropyl cellulose, hydroxypropylmethyl cellulose, crystalline cellulose, starch, polyvinylpyrrolidone, gum arable powder, gelatin, pullulan, low-substituted hydroxypropyl cellulose and the like. Such “disintegrants” include (1) crospovidone, (2) what is called super-disintegrants such as crosscarmellose sodium (FMC-Asahi Chemical) and carmellose calcium (Gotoku Yakuhin) etc., (3) carboxymethyl starch sodium (e.g., product of Matsutani Chemical), (4) low-substituted hydroxypropyl cellulose (e.g., product of Shin-Etsu Chemical), (5) corn starch, and so forth. Said “crospovidone” may be any crosslinked polymer having the chemical name 1-ethenyl-2-pyrrolidinone homopolymer, including polyvinylpyrrolidone (PVP) and 1-vinyl-2-pyrrolidinone homopolymer, and is exemplified by Colidon CL (produced by BASF), Polyplasdon XL (produced by ISP), Polyplasdon XL-IO (produced by ISP), Polyplasdon INF-10 (produced by ISP) and the like. Such “water-soluble polymers” include, for example, ethanol-soluble water-soluble polymers [e.g., cellulose derivatives such as hydroxypropyl cellulose (hereinafter also referred to as HPC) etc, polyvinylpyrrolidone and the like], ethanol-insoluble water-soluble polymers [e.g., cellulose derivatives such as hydroxypropylmethyl cellulose (hereinafter also referred to as HPMC) etc., methyl cellulose, carboxymethyl cellulose sodium and the like, sodium polyacrylate, polyvinyl alcohol, sodium alginate, guar gum and the like] and the like. Such “basic inorganic salts” include, for example, basic inorganic salts of sodium, potassium, magnesium and/or calcium. Such “solvents” include, for example, water for injection, alcohol, propylene glycol, macrogol, sesame oil, corn oil, olive oil and the like. Such “dissolution aids” include, for example, polyethylene glycol, propylene glycol, D-mannitol, benzyl benzoate, ethanol, trisaminomethane, cholesterol, triethanolamine, sodium carbonate, sodium citrate and the like. Such “suspending agents” include, for example, surfactants such as stearyltriethanolamine, sodium lauryl sulfate, laurylaminopropionic acid, lecithin, benzalkonium chloride, benzethonium chloride, glyceryl monostearate etc; hydrophilic polymers such as polyvinyl alcohol, polyvinylpyrrolidone, carboxymethyl cellulose sodium, methyl cellulose, hydroxymethyl cellulose, hydroxyethyl cellulose, hydroxypropyl cellulose etc., and the like. Such “isotonizing agents” include, for example, glucose, D-sorbitol, sodium chloride, glycerol, D-mannitol and the like. Such “buffers” include, for example, buffer solutions of phosphates, acetates, carbonates, citrates etc, and the like. Such “soothing agents” include, for example, benzyl alcohol and the like. Such “preservatives” include, for example, p-oxybenzoic acid esters, chlorobutanol, benzyl alcohol, phenethyl alcohol, dehydroacetic acid, sorbic acid and the like. Such “antioxidants” include, for example, sulfites, ascorbic acid, [alpha]-tocopherol and the like. Such “coloring agents” include, for example, food colors such as Food Color Yellow No. 5, Food Color Red No. 2, Food Color Blue No. 2 etc.; food lake colors, red oxide and the like. Such “sweetening agents” include, for example, saccharin sodium, dipotassium glycyrrhizinate, aspartame, stevia, thaumatin and the like. Such “souring agents” include, for example, citric acid (citric anhydride), tartaric acid, malic acid and the like. Such “bubbling agents” include, for example, sodium bicarbonate and the like. Such “flavorings” may be synthetic substances or naturally occurring substances, and include, for example, lemon, lime, orange, menthol, strawberry and the like.

The P-CAB for use according to the present invention may optionally be used in combination with one or more additional pharmacologically active substances, optionally prepared as a single pharmaceutical composition or as separate preparations to be administered simultaneously or in a sequential manner. Such additional active substances may, for example, be indicated for the treatment and/or symptom control of GERD and/or co-morbidities thereof, such as H. pylori infection. Examples include antacids and antibiotics. The P-CAB may also be used in combination with aspirin or a non-steroidal antiinflammatory drug (NSAID), wherein the P-CAB may be used to prevent and/or reduce gastric-acid related side-effects of the aspirin or NSAID. Examples of suitable NSAIDs include aspirin, indomethacin, ibuprofen, mefenamic acid, diclofenac, etodorac, piroxicam, celecoxib, flurbiprofen, ketoprofen, meloxicam and naproxen, although other NSAIDs would be well known to the skilled person. Where aspirin is used in such a combination with a P-CAB, the aspirin may, for example, be employed for its antiplatelet effects. The combination compositions described herein form another aspect of the invention.

According to the present invention, where a P-CAB and an NSAID are used in combination, the P-CAB and the NSAID may be mixed together and prepared as a single pharmaceutical composition (e.g., tablets, powders, granules, capsules (including soft capsules), liquids, injections, suppositories, sustained-release preparations, etc.) according to a method known per se for combined use, or may also be prepared as separate pharmaceutical compositions and administered to the same subject simultaneously or in a sequential or staggered manner.

The details of the invention, its objectives and advantages are illustrated below in greater detail by the following examples, which are not to be construed as limiting the invention in any way.

### Formulation Example

The preparation of TAK-438 tablets containing 10 mg and 20 mg TAK-438 (as fumarate) per tablet is described in ‘Preparation Examples 1, 2, 3 and 4’ of WO 2014/133059 A1. More specifically, these Preparation Examples describe tablets containing TAK-438 fumarate (i.e. vonoprazan fumarate), denoted ‘Compound A’ therein, at 10 mg TAK-438 per tablet (Preparation Examples 1 and 2, paragraphs [0093] to [0099]) and 20 mg TAK-438 per tablet (Preparation Examples 3 and 4, paragraphs [0100] to [0105]). The contents of WO 2014/133059 A1 are hereby incorporated herein by reference in their entirety, and in particular the contents of Preparation Examples 1 to 4, as identified above.

Table 1 shows the composition of exemplary TAK-438 Tablets which are used for clinical studies. The active ingredient TAK-438 is formulated as the fumarate. The TAK-438 tablet label amount (40 mg) is expressed as the free base.

TABLE 1
Composition of TAK-438 Tablets (40 mg)

| | Components | Quantity per Tablet (mg) |
|---|---|---|
| Core | TAK-438* | 53.44 |
| Tablet | (As the free base) | (40) |
| | Mannitol | 124.54 |
| | Microcrystalline Cellulose | 22 |
| | Hydroxypropyl Cellulose | 6.6 |
| | Fumaric acid | 0.22 |
| | Croscarmellose Sodium | 11 |
| | Magnesium Stearate | 2.2 |
| | Purified Water** | q.s. |
| Film | Hypromellose 2910*** | 6.75 |
| Coating | Polyethylene Glycol 8000*** | 1.5 |
| Solution | Titanium Dioxide*** | 0.75 |
| | Ferric Oxide, Yellow*** | 0.075 |
| | Ferric Oxide, Red*** | 0.075 |
| | Purified Water** | q.s. |
| TOTAL | | 229.15 |

*The molecular weight conversion factor of TAK-438 fumarate to TAK-438 is 461.46/345.39 = 1.336
**Removed during processing
***These ingredients are components of OPADRY Red 03F45081 and OPADRY Yellow 03F42240 (premixed coating materials)

### Description of Manufacturing Process and Process Controls

### Manufacturing Process

1. A binder solution is prepared by dissolving Fumaric Acid and Hydroxypropyl Cellulose in Purified Water by stirring.
2. TAK-438, Mannitol and Microcrystalline Cellulose are charged in a fluidized bed granulator.
3. The charged powders are granulated by spraying the binder solution in the fluid bed granulator. The granules are dried in the fluid bed granulator.
4. The dried granules are milled through a screening mill, or alternatively sieved through a suitable screen.
5. The milled granules are blended with Croscarmellose Sodium and Magnesium Stearate in a diffusion mixer.
6. The blended granules are compressed into tablets by using a tablet press.
7. The tablets are coated with an aqueous film coating solution containing Hypromellose 2910, Polyethylene Glycol 8000, Titanium Dioxide, Ferric Oxide, Yellow and Ferric Oxide, Red by a pan coating.
8. The film coated tablets are inspected visually or by an automated inspection machine.
9. The inspected film coated tablets are packed into a suitable container.

### Reference Example—Clinical Study in PPI-Resistant EE Patients

This was a phase 3, randomized, double-blind, parallel-group, multicenter study to evaluate the dose-response relationships of the acid-inhibitory effect and efficacy of TAK-438 (20 mg and 40 mg) in patients with PPI-resistant EE of LA Classification Grades A to D.

A patient with PPI-resistant EE was defined as a patient who had EE of LA Classification Grades A to D endoscopically confirmed at the examination in the run-in period after receiving a regular or higher PPI dose for at least 8 weeks until immediately before the start of the run-in period. This study consisted of a 7- to 14-day run-in period and an 8-week treatment period. Subjects who entered the run-in period orally received 1 capsule of the PPI lansoprazole 30 mg once daily after breakfast for at least 7 days, but not more than 14 days. As a rule, subjects underwent endoscopic examination 2 days before the end of the run-in period, after receiving the study medication for the run-in period for at least 5 days. The acid-inhibitory effect of the study medication during the run-in period was evaluated by monitoring gastroesophageal pH for 24 hours, beginning from the day before the end of the run-in period. The subject was then randomized at a 1:1 ratio to receive either TAK-438 20 mg or 40 mg and entered the treatment period. Subjects orally received the assigned medications once daily after breakfast for 8 weeks from the day after the end of the run-in period.

### Number of Subjects:

Planned: 20 subjects (10 subjects in each treatment group). Enrolled in the treatment period: 19 subjects

### Diagnosis and Main Criteria for Inclusion:

Subject eligibility for this study required that the subject had received a regular or higher dose of PPI for EE treatment for at least 8 weeks until immediately before the start of the run-in period. In addition, the subject had EE for which a regular or higher dose of PPI had not been effective, or more specifically, the subject had EE of LA Classification Grades A to D endoscopically confirmed at the examination in the run-in period.

### Duration of Treatment:

Run-in period, 7 to 14 days. Treatment period, 8 weeks

### Criteria for Evaluation:

Efficacy: The primary endpoint was the time course of gastroesophageal pH changes over 24 hours at steady state in the treatment period. The primary measure was gastric and esophageal pH 4 holding time ratios (HTRs). Other measures were gastric and esophageal pH 1, 2, 3, 5, 6, and 7 HTRs, mean gastric pH, and mean esophageal pH.

The secondary endpoint was the EE healing rate after 8-week treatment with TAK-438.

Symptomatic aspects of GERD were not examined.

### Summary of Results

The number of subjects with each LA Classification Grade of EE was comparable between the treatment groups at baseline: Grades A/B, 6 subjects in the TAK-438 20 mg group and 7 subjects in the TAK-438 40 mg group; Grades C/D, 3 subjects in each treatment group.

In the treatment period, 9 subjects were allocated to the TAK-438 20 mg group and 10 subjects were allocated to the TAK-438 40 mg group. Of the 19 subjects who were randomized, 18 (94.7%) completed the 8-week study drug administration for the treatment period. One subject in the TAK-438 40 mg group prematurely discontinued the study drug during the treatment period.

### Efficacy Results:

After 2 weeks of TAK-438 administration, the increases in gastric and esophageal pH 4 HTRs were greater in the TAK-438 40 mg group than in the TAK-438 20 mg group, although the differences between the treatment groups were not statistically significant.

The mean 24-hour gastric pH 4 HTR increased to 100.00% in the TAK-438 40 mg group and to 96.46% in the TAK-438 20 mg group; the lower limits of the 95% CI of the mean changes from baseline were greater than 0 in both treatment groups, indicating that the increases in gastric pH 4 HTR were statistically significant. In both treatment groups, the mean gastric pH 4 HTRs increased during both the daytime and nighttime with statistical significance and the changes from baseline were greater during the nighttime than during the daytime. The mean 24-hour esophageal pH 4 HTR increased to 99.86% in the TAK-438 40 mg group and to 98.41% in the TAK-438 20 mg group, although the changes from baseline were not statistically significant in either treatment group.

The mean 24-hour gastric pH 1 HTR was 100.00% in both treatment groups at baseline and after 2 weeks of TAK-438 administration. The mean 24-hour gastric pH 2, 3, 5, 6, and 7 HTRs increased from baseline in both treatment groups. The mean 24-hour esophageal pH 1, 2, 3, 6, and 7 HTRs remained almost unchanged in both treatment groups, while the mean 24-hour esophageal pH 5 HTR slightly increased.

In both treatment groups, the mean gastric pH increased from baseline in the 12-hour and 24-hour periods. The mean esophageal pH also increased, although to a lesser extent than the mean gastric pH.

The EE healing rate after 8-week treatment with TAK-438 was 44.4% (4 out of 9 subjects) in the TAK-438 20 mg group and 55.6% (5 out of 9 subjects) in the TAK-438 40 mg group.

### CONCLUSIONS

The administration of TAK-438 20 mg or 40 mg suppressed gastric acid secretion in subjects with PPI-resistant EE during the nighttime as well as during the daytime.

TAK-438 seemed to be effective, safe, and well tolerated in subjects with PPI-resistant EE of LA Classification Grades A to D.

### Clinical Example of Invention—Clinical Study in PPI Partial Responders

A study is held to evaluate TAK-438 (20 mg QD and 40 mg QD) in subjects who have a history of heartburn-predominant GERD despite an adequate course of PPI treatment and who are then confirmed to have a partial response to a 4-week treatment course with a PPI (esomeprazole 40 mg QD).

Subjects are eligible for participation in the study if they:
have a documented history of symptoms of both heartburn and acid regurgitation prior to entry into the study;
have a history of heartburn-predominant GERD despite an adequate course of PPI treatment;
continue to have symptoms of heartburn (with or without regurgitation) following 4 weeks treatment with a high dose of esomeprazole 40 mg QD;
have symptoms of heartburn (with or without regurgitation) which increase following a 2-week washout period compared with the prior 4 week course of high dose esomeprazole 40 mg QD.

The subjects entered into the study are NERD and mild (LA grade A, as defined by the Los Angeles Classification) erosive esophagitis (EE).

The study includes the following periods:
an initial 1-week general screening period during which the subject remains on their prescribed PPI
a 4-week treatment period with esomeprazole 40 mg QD
a 2-week placebo off-PPI assessment washout period
subjects who remained symptomatic are randomized to a 4-week treatment period with either TAK-438 (20 mg QD or 40 mg QD) or esomeprazole (40 mg QD)

Subjects are trained in how to complete a symptom diary so that they can accurately record their daytime and nighttime symptoms during the study. (RESQ-eD questionnaire in Vakil et al., Clinical and Translational Gastroenterology 3, e7, 2012).

Esomeprazole is chosen for the study as it is considered the current gold standard PPI for the treatment of GERD. 40 mg is the highest approved dose, and likely to be the most appropriate dose for this difficult-to-treat population.

The study involves subjects who have a documented history of symptoms of both heartburn and acid regurgitation prior to entry into the study, and of not responding fully to their PPI treatment (at least 2 months of heartburn predominant troublesome symptoms after treatment at the maximum approved dose of PPI), and who have a partial response to treatment with a high dose of esomeprazole 40 mg QD (defined as having heartburn on 2 to 5 days of the last week of a 4 week run-in with esomeprazole 40 mg). To further confirm that their heartburn is acid-related, subjects also have to have an increase of at least 2 symptom days of heartburn (with or without regurgitation) in the last week of a 2-week off-PPI assessment period (4 to 7 symptom days) compared with the last week of the PPI assessment period. Including the washout-after-PPI treatment results in a higher pre-randomisation baseline heartburn frequency allowing the effects of both treatments as well as the treatment difference to be estimated.

The study shows that the P-CAB TAK-438 can reduce heartburn and/or regurgitation symptoms throughout the whole day and night, and can increase the percentage of heartburn-free days and nights (in 24-hour periods) in PPI partial responders. The P-CAB can potentially increase the proportion of PPI partial responder patients who experience one or more sustained resolutions of heartburn during the period of treatment, wherein a sustained resolution is classed as a continuous period of seven or more days without daytime or nighttime heartburn. These results show that P-CABs, including TAK-438, are effective for use in the reduction of GERD symptoms in patients with an insufficient response to a proton pump inhibitor (PPI), and in particular for the treatment, prevention and/or reduction of symptoms of non-erosive reflux disease (NERD) and erosive esophagitis (EE) of Grade A. The ability of P-CABs to achieve these effects in PPI partial responders is unexpected, given the earlier published trials teaching that clinical differences between P-CABs and PPIs would be limited in this difficult-to-treat patient population.

1. A method for treating, preventing-and/or reducing gastro-esophageal reflux disease (GERD) symptoms in a patient who is a partial responder to a proton pump inhibitor (PPI), the method comprising administering a dose of a potassium-competitive acid blocker (P-CAB) to the patient.

2. The method of claim 1, wherein GERD is non-erosive reflux disease (NERD) as defined by the Los Angeles (LA) Classification.

3. The method of claim 1, wherein GERD is erosive esophagitis (EE) of Grade A as defined by the LA Classification.

4. The method of claim 1, wherein the P-CAB is 1-[5-(2-fluorophenyl)-1-(pyridin-3-ylsulfonyl)-1H-pyrrol-3-yl]-N-methylmethanamine (vonoprazan), revaprazan (YH1855), YH4808, RQ-4, CS-526, RQ-774, soroprazan (BY359), AZD0865, SCH 28080, a salt thereof, or combination thereof.

5. The method of claim 4, wherein the P-CAB is vonoprazan or a salt thereof.

6. The method of claim 5, wherein the P-CAB is vonoprazan fumarate.

7. The method of claim 1, wherein the dose of the P-CAB administered is 0.5 mg to 500 mg per day.

8. The method of claim 7, wherein the dose of the P-CAB is 20 mg to 40 mg per day.

9. The method of claim 8, wherein the dose of the P-CAB is 20 or 40 mg per day.

10. The method of claim 1, wherein the PPI is lansoprazole, pantoprazole, omeprazole, rabeprazole, dexlansoprazole, esomeprazole,—an optically active form thereof, a salt thereof, or combination thereof.

11. The method of claim 10, wherein the PPI is dexlansoprazole, esomeprazole, or a salt thereof.

12. The method of claim 11, wherein the PPI is esomeprazole or a salt thereof.

13. The method of claim 1, wherein the P-CAB is administered by once-daily dosing.

14. The method of claim 1, wherein the P-CAB is 1-benzyl-7-[N-(4-fluorobenzyl)-N-methyl]amino-2,3-dimethyl-1H-pyrrolo[2,3-c]pyridine hydrochloride; 1-(3-fluorobenzyl)-7-(4-fluorobenzylamino)-2,3-dimethyl-1H-pyrrolo[2,3-c]pyridine hydrochloride; 7-(4-fluorobenzylamino)-1-isobutyl-2,3-dimethyl-1H-pyrrolo[2,3-c]pyridine hydrochloride; 11-isobutyl-2,3-dimethyl-7-(2-methylbenzylamino)-1H-pyrrolo[2,3-c]pyridine hydrochloride; 7-(4-chlorobenzylamino)-1-(4-fluorobenzyl)-2,3-dimethyl-1H-pyrrolo[2,3-c]pyridine hydrochloride; 7-(4-chlorobenzylamino)-2,3-dimethyl-1-propyl-1H-pyrrolo[2,3-c]pyridine hydrochloride; 2,3-dimethyl-7-(naphthalen-2-yl)-1H-pyrrolo[2,3-c]pyridine hydrochloride; 7-(1,2,3,4-tetrahydroisoquinolin-2-yl)-2,3-dimethyl-1H-pyrrolo[2,3-c]pyridine hydrochloride; 1-benzyl-7-(1,2,3,4-tetrahydroisoquinolin-2-yl)-2,3-dimethyl-1H-pyrrolo[2,3-c]pyridine hydrochloride; 7-(1,2,3,4-tetrahydroisoquinolin-2-yl)-1,2,3-trimethyl-1H-pyrrolo[2,3-c]pyridine hydrochloride; 1-propyl-7-(1,2,3,4-tetrahydroisoquinolin-2-yl)-2,3-dimethyl-1H-pyrrolo[2,3-c]pyridine hydrochloride; 1-(2-methylthiazol-4-ylmethyl)-7-(1,2,3,4-tetrahydroisoquinolin-2-yl)-2,3-dimethyl-1H-pyrrolo[2,3-c]pyridine hydrochloride; 1-(4-methylbenzyl)-7-(1,2,3,4-tetrahydroisoquinolin-2-yl)-2,3-dimethyl-1H-pyrrolo[2,3-c]pyridine hydrochloride; 1-(3-fluorobenzyl)-7-(1,2,3,4-tetrahydroisoquinolin-2-yl)-2,3-dimethyl-1H-pyrrolo[2,3-c]pyridine hydrochloride; 1-(4-methoxybenzyl)-7-(1,2,3,4-tetrahydroisoquinolin-2-yl)-2,3-dimethyl-1H-pyrrolo[2,3-c]pyridine hydrochloride; 1-allyl-7-(1,2,3,4-tetrahydroisoquinolin-2-yl)-2,3-dimethyl-1H-pyrrolo[2,3-c]pyridine hydrochloride; 1-(4-tert-butylbenzyl)-7-(1,2,3,4-tetrahydroisoquinolin-2-yl)-2,3-dimethyl-1H-pyrrolo[2,3-c]pyridine hydrochloride; 1-(2-vinyloxyethyl)-7-(1,2,3,4-tetrahydroisoquinolin-2-yl)-2,3-dimethyl-1H-pyrrolo[2,3-c]pyridine hydrochloride; 2-(1-allyl-2,3-dimethyl-1H-pyrrolo[2,3-c]pyridin-7-yl)-1-methyl-1,2,3,4-tetrahydroisoquinoline hydrochloride; 2-[1-(3-fluorobenzyl)-2,3-dimethyl-1H-pyrrolo[2,3-c]pyridin-7-yl]-1-methyl-1,2,3,4-tetrahydroisoquinoline hydrochloride; 2-(1-allyl-2,3-dimethyl-1H-pyrrolo[2,3-c]pyridin-7-yl)-6-fluoro-1-methyl-1,2,3,4-tetrahydroisoquinoline hydrochloride; alternative salt thereof; or combination thereof.

15. The method of claim 14, wherein the P-CAB is 1-benzyl-7-(1,2,3,4-tetrahydroisoquinolin-2-yl)-2,3-dimethyl-1H-pyrrolo[2,3-c]pyridine hydrochloride; 1-(3-fluorobenzyl)-7-(1,2,3,4-tetrahydroisoquinolin-2-yl)-2,3-dimethyl-1H-pyrrolo[2,3-c]pyridine hydrochloride; or alternate salt thereof.

16. The method of claim 1, wherein the P-CAB is (S)-(−)-4-[(5,7-difluoro-3,4-dihydro-2H-chromen-4-yl)oxy]-N,N,2-trimethyl-1H-benzimidazole-6-carboxamide; (S)-(−)-4-[(5,7-difluoro-3,4-dihydro-2H-chromen-4-yl)oxy]-2-methyl-6-(pyrrolidin-1-ylcarbonyl)-1H-benzimidazole; (S)-(−)-4-[(5-fluoro-3,4-dihydro-2H-chromen-4-yl)oxy]-N,N,2-trimethyl-1H-benzimidazole-6-carboxamide; (−)-1-(2-methoxyethyl)-N,N,2-trimethyl-8-phenyl-1,6,7,8-tetrahydrochromeno[7,8-d]imidazole-5-carboxamide; (+8-(4-fluorophenyl)-1-(2-methoxyethyl)-N,N,2-trimethyl-1,6,7,8-tetrahydrochromeno[7,8-d]imidazole-5-carboxamide; 8-(4-fluorophenyl)-1-(3-hydroxypropyl)-N,N,2-trimethyl-1,6,7,8-tetrahydrochromeno[7,8-d]imidazole-5-carboxamide; 8-(4-fluorophenyl)-1-(isoxazol-3-ylmethyl)-N,N,2-trimethyl-1,6,7,8-tetrahydrochromeno[7,8-d]imidazole-5-carboxamide; 8-(4-fluorophenyl)-N-(2-hydroxyethyl)-1-(2-methoxyethyl)-N,2-dimethyl-1,6,7,8-tetrahydrochromeno[8,7-d]imidazole-5-carboxamide; (8-(4-fluorophenyl)-1-(2-methoxyethyl)-2-methyl-1,6,7,8-tetrahydrochromeno[8,7-d]imidazol-5-yl)(morpholino)methanone; 1-{5-(2-fluorophenyl)-1-[(6-methylpyridin-3-yl)sulfonyl]-1H-pyrrol-3-yl}-N-methylmethanamine; 1-[4-fluoro-5-phenyl-1-(pyridin-3-ylsulfonyl)-1H-pyrrol-3-yl]-N-methylmethanamine; N-methyl-1-[5-(4-methyl-3-thienyl)-1-(pyridin-3-ylsulfonyl)-1H-pyrrol-3-yl]methanamine; 1-[5-(2-fluorophenyl)-1-(pyridin-3-ylsulfonyl)-1H-pyrrol-3-yl]-N-methylmethanamine; N-Methyl-1-[5-(2-methylphenyl)-1-(pyridine-3-ylsulfonyl)-1H-pyrrol-3-yl]methanamine; 8-[{2,6-dimethylbenzyl}amino]-N-[2-hydroxyethyl]-2,3-dimethylimidazo[1,2-a]pyridine-6-carboxamide; 8R,9R)-7-(2-methoxyethoxy)-2,3-dimethyl-9-phenyl-7,8,9,10-tetrahydroimidazo[1,2-h][1,7]naphthyridine-8-ol; 5,6-dimethyl-2-(4-fluoro-phenylamino)-4-(1-methyl-1,2,3,4-tetrahydroisoquinoline-2-yl)pyrimidine; (S)-(−)-N,N,2,3-tetramethyl-8-O-tolyl-3,6,7,8-tetrahydrochromeno[7,8-d]imidazole-5-carboxamide; 7-(4-fluorobenzyloxy)-2,3-dimethyl-1-{[(1S,2S)-2-methyl cyclopropyl]methyl}-1H-pyrrolo[2,3-d]pyridazine; suitable salt thereof, or combination thereof.

17. The method of claim 16, wherein the P-CAB is (S)-(−)-4-[(5,7-difluoro-3,4-dihydro-2H-chromen-4-yl)oxy]-N,N,2-trimethyl-1H-benzimidazole-6-carboxamide; (−)-1-(2-methoxyethyl)-N,N,2-trimethyl-8-phenyl-1,6,7,8-tetrahydrochromeno[7,8-d]imidazole-5-carboxamide; 8-[{2,6-dimethylbenzyl}amino]-N-[2-hydroxyethyl]-2,3-dimethylimidazo[1,2-a]pyridine-6-carboxamide; or suitable salt thereof.
